(19) United States
(12) Patent Application Publication
(10) Pub. No.: US 2022/0110785 A1
(43) Pub. Date: Apr. 14, 2022

(54) THERAPEUTIC DEVICE Applying COMPRESSION AND HEAT TREATMENT

(71) Applicant: Reetu Sharma, Granite Bay, CA (US)

(72) Inventor: Reetu Sharma, Granite Bay, CA (US)

(21) Appl. No.: 17/499,745

(22) Filed: Oct. 12, 2021

Related U.S. Application Data
(60) Provisional application No. 63/090,469, filed on Oct. 12, 2020.

(51) Int. Cl. A61F 7/02; A61H 23/00

(52) U.S. Cl. CPC A61F 7/02; A61F 2007/0042; A61F 2007/0295; A61H 23/00

(57) ABSTRACT

A device applies therapeutic treatment to a subject. The therapeutic treatment includes any two modalities applied simultaneously, the modalities selected from compression, heat, vibration, and massage. The device includes a main body; hinges coupled to the main body on each side of the main body; and respective plates connected to the hinges, wherein the respective plates contact opposing surfaces of a body.

### CROSS REFERENCE TO RELATED APPLICATIONS

This application claims priority under 35 U.S.C. § 119(e) to U.S. Provisional Application No. 63/090,469, filed Oct. 12, 2020, which is hereby incorporated by reference in its entirety.

### BACKGROUND

Pain has been a long standing debilitating condition that interferes with quality of life by affecting work, sleep, physical and mental wellbeing, and social interactions. Pain has been tied to depression, anxiety and other major medical conditions. Unfortunately, the panacea of pain management has yet to be discovered. Furthermore, pharmaceutical regulations and concerns are catalysts that have triggered increased urgency to develop non-pharmaceutical pain management solutions. Particularly, in July 2015, the FDA asked that both prescription and over the counter (OTC) OTC nonsteroidal anti-inflammatory drugs (NSAIDs) strengthen their warning labels to indicate the potential risk of heart attacks and strokes, the risk increasing with higher doses. The possibility of developing kidney failure and bleeding ulcers further exacerbates the risks. Meanwhile, the “opioid epidemic” has deterred prescriptions of narcotic pain medications due to concerns of addiction.

The gate theory of pain management is a widely accepted theory of how and why we perceive pain. In short, a gate at the level of the spinal cord allows signals of pain to be relayed up to the brain for “interpretation.” Three factors influence the state and degree of opening and closing of the pain gate. The strength of the noxious stimulus opens the pain gate. The strength of counterstimuli (also relayed by sensory nerves) including pressure, touch, temperature, vibration sensation closes the pain gate. Finally, the brain itself can open or close the pain gate. These factors explain why distraction or hypnosis can work for pain control and why depressed mood can worsen the perception of pain. The gate theory has lent credence to techniques of pain management such as The TENS unit, acupuncture and massage, among others.

### SUMMARY

Described herein, in some embodiments, is a device that applies therapeutic treatment to a subject, wherein the therapeutic treatment comprises any two modalities applied simultaneously, the modalities selected from compression, heat, vibration, and massage. The device includes: a main body; hinges coupled to the main body on each side of the main body; and respective plates connected to the hinges, wherein the respective plates are configured to contact opposing surfaces of a body. In some embodiments, opposing surfaces may refer to complementary surfaces and/or oppositely disposed surfaces. For example, opposing surfaces may include a right portion of a left thigh and a left portion of a left thigh. Another set of opposing surfaces may include a front of a knee and a back of a knee. Another set of opposing surfaces may include a stomach region and a back.

In some embodiments, the respective plates include heat sources embedded within; and the device applies heat and compression simultaneously.

In some embodiments, the heat sources provide heat at between 39 degrees and 42 degrees Celsius for periods of between five and ten minutes.

In some embodiments, the compression is applied at between 20 mm Hg and 120 mm Hg.

In some embodiments, the plates apply vibration simultaneously with heat, wherein the vibration is between 20 hz and 155 hz.

In some embodiments, the device further includes respective arms disposed between the hinges and the plates, wherein the arms are translatable with respect to the hinges in a direction perpendicular to an alignment of the device, so that when the arms are translated with respect to the hinges, the device applies asymmetric compression.

In some embodiments, the arms each include a ledge; the hinges each comprise a gear embedded within; and each respective ledge contacts a gear and locks at discrete positions.

In some embodiments, the hinges each include a pawl embedded within, the pawl engaging teeth of the gear at discrete intervals.

In some embodiments, the device is torsioned at an angle of between 20 degrees and 45 degrees between the respective plates.

In some embodiments, the therapeutic treatment includes compression, heat, vibration, and massage.

In some embodiments, the vibration includes at least two types of simultaneous or sequential vibration selected from a group consisting of: oscillation vibration, spiral vibration, and triplanar vibration.

In some embodiments, the vibration has a frequency of under 200 Hz.

In some embodiments, the therapeutic treatment includes a bidirectional massage.

Various embodiments of the present disclosure provide a method and/or non-transitory storage medium implemented by a device as described above.

Described herein, in some embodiments, is a method of applying a therapeutic treatment to a subject, wherein the therapeutic treatment includes any two modalities applied simultaneously, the modalities selected from compression, heat, vibration, and massage. The therapeutic treatment may be applied using a device that includes a main body, hinges coupled to the main body on each side of the main body; and respective plates connected to the hinges, The application of the therapeutic treatment includes contacting the plates to opposing surfaces of a body.

In some embodiments, the method further includes generating heat from heat sources embedded within the device; and the application of the therapeutic treatment comprises applying heat and compression simultaneously.

In some embodiments, the application of the therapeutic treatment includes applying the heat at between 39 degrees and 42 degrees Celsius for period of between five and ten minutes.

In some embodiments, the application of the therapeutic treatment includes applying the compression at between 20 mm Hg and 120 mm Hg.

In some embodiments, the application of the therapeutic treatment includes applying heat simultaneously with vibration, wherein the vibration is between 20 hz and 155 hz.

In some embodiments, the therapeutic treatment includes an asymmetric compression, and the application of the asymmetric compression includes translating arms disposed between the hinges and the plates, the translation being in a direction perpendicular to an alignment of the device.

In some embodiments, the translation of the arms includes contacting a gear with a ledge to lock the ledge at discrete positions, wherein the ledge is comprised within one of the arms and the gear is embedded within one of the ledges.

In some embodiments, the locking of the ledge includes engaging, with a pawl, teeth of the gear at discrete intervals corresponding to the discrete positions, the pawl being embedded within the hinges.

In some embodiments, the method further includes torsioning the device at an angle of between 20 degrees and 45 degrees between the respective plates.

In some embodiments, the therapeutic treatment comprises compression, heat, vibration, and massage.

These and other features of the devices, methods, and non-transitory computer readable media disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for purposes of illustration and description only and are not intended as a definition of the limits of the invention.

### BRIEF DESCRIPTION OF THE DRAWINGS

Certain features of various embodiments of the present technology are set forth with particularity in the appended claims. A better understanding of the features and advantages of the technology will be obtained by reference to the following detailed description that sets forth illustrative embodiments, in which the principles of the invention are utilized, and the accompanying drawings of which:

FIGS. 1A-1G illustrate an exemplary device that relieves pain and/or implements a therapeutic treatment, in accordance with an embodiment. In particular, the exemplary device illustrated in FIGS. 1A-1G may implement asymmetric compression.

FIG. 2 illustrates another exemplary device that relieves pain and/or implements a therapeutic treatment, in accordance with an embodiment.

FIGS. 3A-3D illustrate exemplary implementations of the device of FIGS. 1A-1E or FIG. 2, in accordance with an embodiment.

FIGS. 4A-4D illustrate an exemplary implementation of a plate of the device of FIGS. 1A-1E or FIG. 2, in accordance with an embodiment.

FIG. 5 illustrates an implementation used in conjunction with the device FIGS. 1A-1E or FIG. 2, in accordance with an embodiment.

FIGS. 6A-6B illustrate implementations used in conjunction with the device FIGS. 1A-1E or FIG. 2, in accordance with an embodiment.

FIGS. 7A-7C illustrate implementations of a processor, in accordance with an embodiment.

FIG. 8 illustrates a block diagram of a computer system 800 upon which any of the embodiments described herein may be implemented.

### DETAILED DESCRIPTION

Embodiments described in this application provide a device that is accessible, portable, and convenient. The device may combine implementations of vibration, heat, compression, and massage treatment to reduce pain and/or discomfort, which would in turn ameliorate a quality of life while reducing or eliminating a reliance on pain medications that may have additional side effects. The simultaneous combination of any of vibration, heat, compression, and massage treatment may together provide a synergistic impact such that a combined treatment including any of the aforementioned modalities may have a larger impact than a sum of individual treatments of vibration, heat, compression, or massage alone. In some examples, a simultaneous combination of heat and compression may be applied simultaneously. Additionally, the device may augment bone remodeling and growth.

Furthermore, the device may even be used in outer space to combat muscle and/or joint atrophy. In some embodiments, the device may also be used to provide therapeutic treatment tailored towards COVID-19. In some embodiments, the device may be manually operated or may be programmable, such as via Bluetooth or other wireless or wired communications, to implement particular settings or sequences of treatment to prevent future episodes of pain or discomfort. Therefore, the device may not need to be replaced, and thus, can unobtrusively operate while a user is engaged in other tasks such as work, sleep, or play.

The device may be secured or clamped at surfaces to provide pain relief and/or therapeutic treatment to tissues underneath the surfaces. The tissues may include bone, joint, or muscle. The device may be adhered, at or near its ends, to different body surfaces, such that the two body surfaces are tightly inserted (“sandwiched”) between the ends of the device to form a tight and secure fit to the body surfaces. The device inhibits pain 1) locally at the level of a tissue, muscle, or joint, 2) at the level of the relay station, such as, at the spinal cord or peripheral nerves, and 3) at the level of the brain or the central nervous system.

In some embodiments, the device may be used for a wide range of users. Optionally, in some embodiments, the device may be tailored to an individual user, receive feedback, and adjust a setting or sequence of treatment based on the individual user's physiological responses such as respiratory rate, heart rate, EMG signals, sleep, and activity patterns. In some embodiments, the device may be programmed to implement particular treatments depending on one or more particular tissues, such as joints, to be targeted. The device can combine vibration treatment, thermotherapy, compression, and/or massage therapy, along with other treatments.

The device may be programmed to provide focused vibration treatment at specific frequencies. The focused vibration targets specific mechanoreceptors and nerve pathways that can active the spinal gate mechanism. Vibration improves range of joint motion while reducing muscle atrophy and joint pain. Studies have demonstrated that vibration provides anabolic mechanical signals to bone, muscles, and tendons. Vibration therapy (VT) has been shown to reduce delayed onset muscle soreness (DOMS) which occurs 48 hours after a workout, to a shorter duration compared to other modalities. VT has been purported to improve balance, strength, and proprioception in the elderly, prevent osteopenia, and potentially reduce a risk of falling. Vibration augments other therapies that are concurrently or simultaneously implemented, such as thermotherapy, compression, and/or massage.

In some embodiments, a frequency of vibration may be at most 200 hz (hertz), or at most 160 hz. In some embodiments, the range of vibration frequencies may be between 20 hz and 155 hz, between 20 hz and 120 hz, between 20 hz and 75 hz, or between 60 and 120 hz, and any other subranges thereof. In some examples, vibration applied to the trunk, including a shoulder or back of a human, may be in a range between 60 hz and 120 hz, while vibration applied to other small or medium areas of a limb, including limbs such as arms and legs, may be between 20 hz and 75 hz. All ranges recited are understood to be inclusive. For example, 20 hz to 120 hz is understood to include 20 hz and 120 hz. In addition, vibration and compression applied simultaneously result in a synergistic effect according to test results. For example, across a population of 38 subjects, an application of vibration lower than 50 hz reduces the amount of pressure applied by 5 to 10 mm Hg, in order to obtain an equivalent impact from compression alone. However, in some applications of higher frequencies of vibration, such as over 50 hz or over 60 hz, a slight increase in pressure, such as up to 5 mm Hg, may be simultaneously applied, compared to the application of pressure alone.

Meanwhile, thermotherapy, or application of heat, increases local blood flow to promote pain relief and healing while reducing resting muscle tone and spasticity. Thermotherapy also reduces DOMS, decreases stiffness and muscle fatigue through vasodilation, relaxes muscles to alleviate pain, increases pain thresholds, improves ranges of motion, and accelerates tissue healing. In addition, heat and compression applied simultaneously result in a synergistic effect according to test results. For example, across a population of 38 subjects, an application of heat at a temperature of between 39 degrees Celsius and 42 degrees Celsius for five to ten minute durations reduces the average applied compression by 10 mm Hg, in order to obtain an equivalent impact from compression alone. In other words, if a subject were treated with 60 mm Hg without heat, the subject may experience a same treatment, therapy, or impact with a treatment of 50 mm Hg simultaneously with the application of heat.

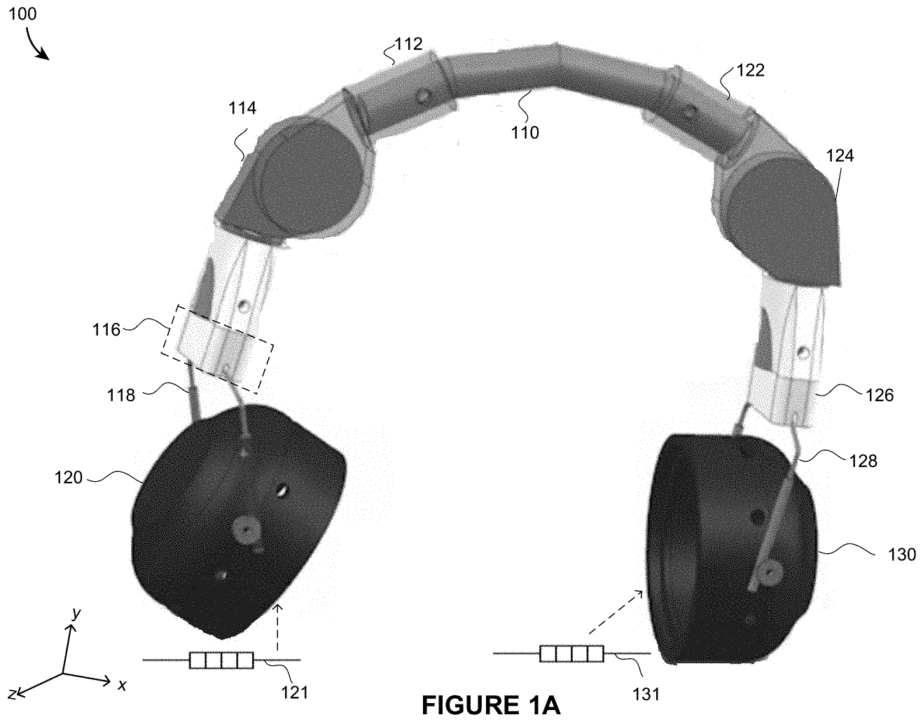

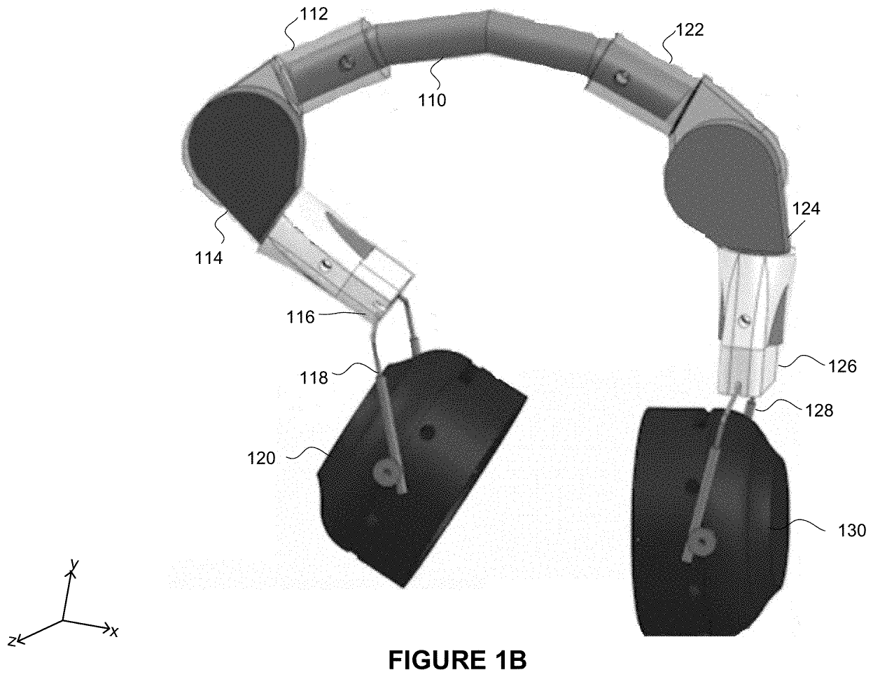

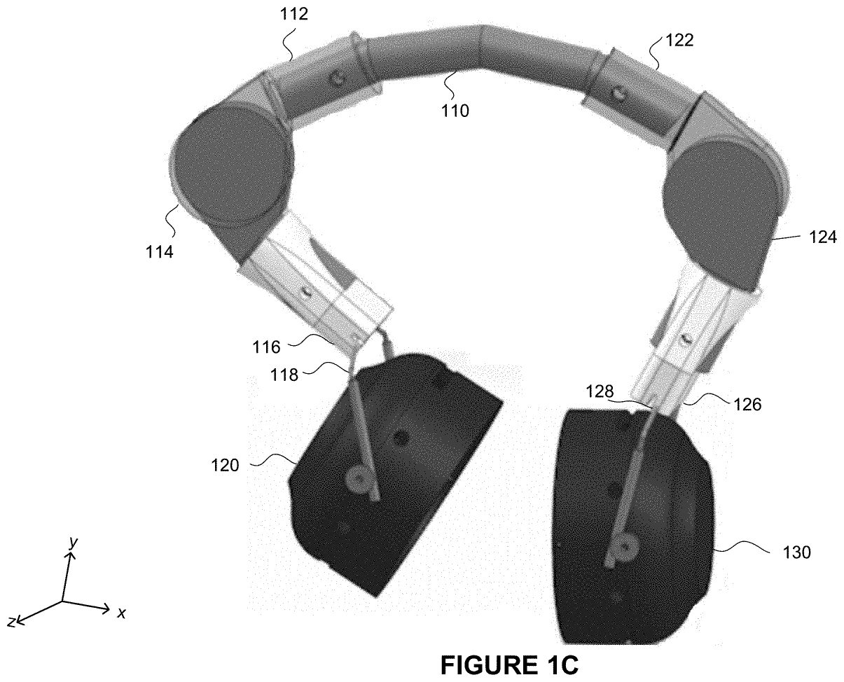

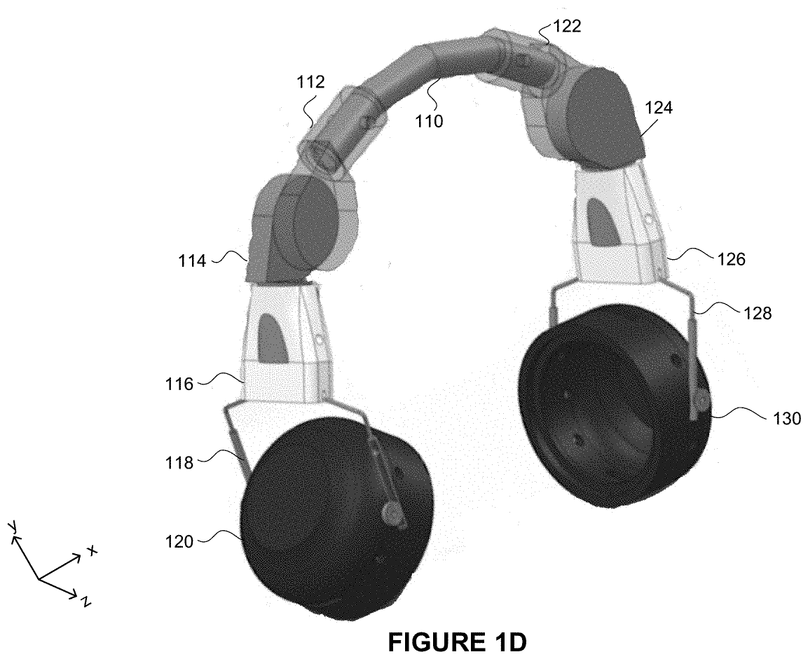

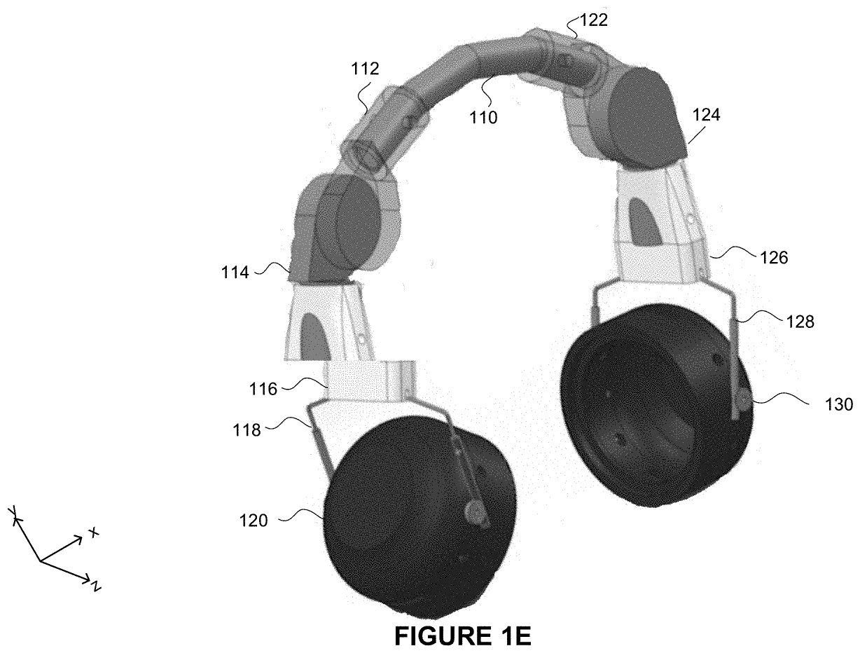

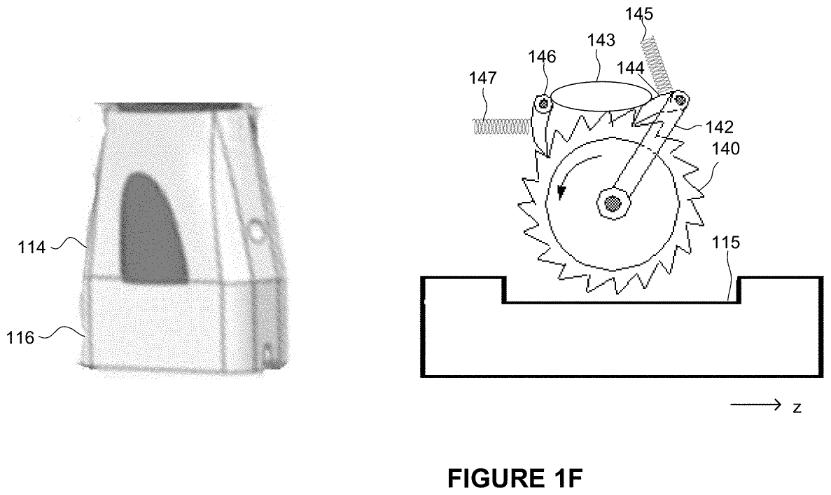

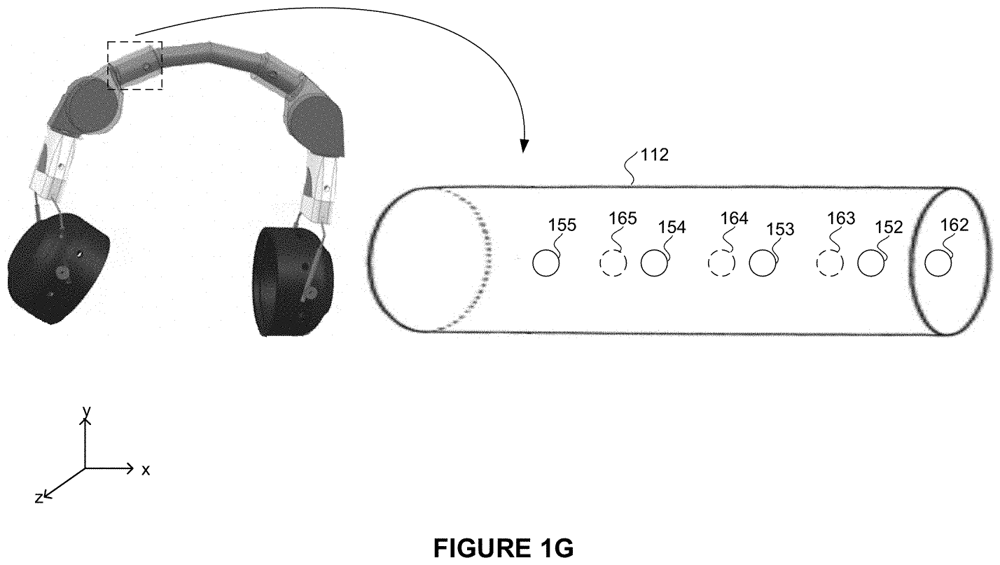

Compression has been shown in increase local blood circulation and assist in lymphatic drainage of muscles. As a result, compression alleviates pain, improves tissue healing, decreases lactic acid accumulation, and improves exercise or workout performance. Compression facilitates an even distribution of heat so that a user or patient may receive a sensation of the heat more evenly and strongly as a result of applying compression simultaneously. In particular, asymmetric compression, as will be further described with respect to FIGS. 1A-1G, has been demonstrated, according to testing results, to result in even further improvements in relaxation of muscles and soft tissue, pain relief, and healing, compared to symmetric compression. In some embodiments, an amount of compression may be at most 120 mm Hg (millimeters mercury), or at most 160 mm Hg. In some embodiments, the range of compressions may be between 20 mm Hg and 160 mm Hg, between 20 mm Hg and 120 mm Hg, between 50 mm Hg and 160 mm Hg, between 60 mm Hg and 120 mm Hg, between 60 mm Hg and 100 mm Hg, between 20 mm Hg and 60 mm Hg, between 20 mm Hg and 40 mm Hg, and any other subranges thereof. In some examples, compression applied to the trunk, including a shoulder or back of a human, may be in a range between 60 mm Hg and 100 mm Hg, while compression applied to other small or medium areas of a limb, including limbs such as arms and legs, may be between 20 mm Hg and 40 mm Hg. All ranges recited are understood to be inclusive. For example, 20 mm Hg to 160 mm Hg is understood to include 20 mm Hg and 160 mm Hg.

Massage therapy promotes relaxation, alleviates perception of anxiety, relaxes muscles, and increases local blood flow, thereby reducing inflammation and accelerating remodeling of injured tissue and recovery through mechanical deformation of tissues. Massaging further improves range of motion due to removal of lactate, which may otherwise cause stiffness. Massaging also causes the brain to release endorphins and promotes release of serotonin, while reducing cortisol release. Overall, massage therapy improves mood, sleep, and appetite while reducing stress.

Test results have demonstrated a hitherto unknown, and somewhat unexpected, synergistic impact when certain modalities, including vibration, heat, compression and massage therapy, are simultaneously applied. In particular, a combination of heat and compression, or a combination of vibration and compression, combine to reinforce the impacts of each other on tissue and muscle healing, comfort, pain relief, and long-term muscle conditioning, such as in an event of a sprain or non-acute injury. A combination of any of the enumerated ranges above may be implemented. For example, a combination of between 20 hz and 60 hz vibration, 39 to 42 degrees Celcius heat application for five to ten minute durations, and between 20 mm Hg and 120 mm Hg compression, along with massage, may be implemented. Exemplary devices that perform such combination therapy and have provided the successful test results are described below.

FIG. 1A illustrates an exemplary device 100 that relieves pain and/or implements a therapeutic treatment, in accordance with an embodiment. In some embodiments, the device 100 may be implemented as a clamp or a brace. In FIG. 1A, the device 100 includes a band or main body (hereinafter “main body”) 110, a first arm 112 that may be connected, coupled, or linked (hereinafter “connected”) to and/or partially enclose the main body 110, a first hinge 114 connected to the first arm 112, a second arm, an attachment, or appendage (hereinafter “second arm”) 116 connected to the first hinge 114, a second hinge 118 connected to the second arm 116, and plates or pads (hereinafter “plates”) 120. In some examples, the first hinge 114 may be spring loaded in order to apply compression. In some examples, the second arm 116 may be part of the first hinge 114. For example, the second arm 116 may be part of a female component or adapter corresponding to a male component or adapter of the first hinge 114. The second arm 116 may be translatable, with respect to the first hinge 114, along any axes, such as along a z-axis. In some examples, the second arm 116 may be rotatable, with respect to the first hinge 114, about a y-axis. In some examples, the plates 120 may be embedded with or otherwise associated with heating components such as a heat source 121. Although the plates 120 are illustrated as being circular in cross-section, in some embodiments, the plates 120 may have other cross-sectional shapes such as squares or rectangles.

Similarly, on an opposite side of the first arm 112, a third arm 122 may partially enclose the main body 110 and/or be connected to the main body 110. A third hinge 124 may be connected to the third arm 122. A fourth arm, a second attachment, or a second appendage 126 may be connected to the third hinge 124. A fourth hinge 128 may be connected to the fourth arm 126. Plates 130 may be connected to the fourth hinge 128. In some examples, the third hinge 124 may be spring loaded in order to apply compression at the plates 120 and 130 when contacting a surface of a body. In some examples, the fourth arm 126 may be part of the third hinge 124. For example, the fourth arm 126 may be part of a female component or adapter corresponding to a male component or adapter of the third hinge 124. The fourth arm 126 may be translatable, with respect to the third hinge 124, along any axes, such as along a z-axis. In some examples, the fourth arm 126 may be rotatable, with respect to the third hinge 124, about a y-axis. In some examples, the plates 130 may be embedded with or otherwise associated with heating components such as a heat source 131. Although the plates 130 are illustrated as being circular in cross-section, in some embodiments, the plates 130 may have other cross-sectional shapes such as squares or rectangles. In other embodiments, instead of the plates 120 and 130, other attachments may be installed instead. For example, the attachments may include cups such as suction cups, or chambers, compartments or containers, and/or adapters that are configured to interface or attach to the cups, chambers, compartments, or containers.

The main body 110 may be made from any of injection molded plastics or metals such as cast alloys and sheet metals. The main body 110 may be flexible to accommodate different orientations or positions of the terminal portions. For example, the main body 110 may be flexible to accommodate a torsion or twisting angle, such as a torsion angle of between 20 and 45 degrees. As a result of the torsion angle, the plates 120 and the plates 130, and/or the main body 110, may be rotated about a y-axis, and/or rotated within a xz-plane. The z-axis may be a direction going out of the page. Applying therapeutic treatment at a twist or torsion angle may implement asymmetric compression on the two plates 120 and 130 and provide a stretching to a an underlying muscle or soft tissue, thereby facilitating or causing the release of endorphins at an accelerated rate compared to an application of therapeutic treatment with no twist or torsion. Additionally, applying a torsion angle may facilitate therapy on slightly different portions of opposing surfaces. The main body 110 may be arcuate, U-shaped, or flat-sectioned.

Any joints between any of the aforementioned components (e.g., between the main body 110 and the first arm 112, between the first arm 112 and the first hinge 114, between the first hinge 114 and the second arm 116, between the second arm 116 and the second hinge 118, or between the second hinge 118 and the plates 120) may be adapted so that one of the components may be rotated and/or translated with respect to another component. For example any of the aforementioned components, such as, between the first arm 112 and the first hinge 114, may have a mechanism similar or same to that illustrated in FIG. 1F, which includes a gear and a pawl so that translation of components may occur at discrete intervals. For example, one component of the aforementioned components may rotate about a y-axis with respect to another directly adjacent component in order to attain a torsion angle. Additionally, each of the aforementioned components may be modular.

FIGS. 1B and 1C illustrate that rotation of the first hinge 114 and/or the third hinge 124 may cause the device 100 to compress, or, reduce a distance between the plates 120 and 130 along a x-axis.

FIGS. 1D and 1E illustrate that the second arm 116 may be translated with respect to a z-axis, which results in a torsion angle between the plates 120 and 130. In FIG. 1E, the second arm 116 has been translated in a z-axis direction with respect to the first hinge 114, compared to FIG. 1D. Thus, the second arm 116 may be translated in a direction perpendicular to an alignment of the device 100. As illustrated in FIG. 1A, the device 100 may be aligned along a xy-plane. A mechanism of the translation is illustrated in FIG. 1F. The second arm 116 may be translated with respect to the first hinge 114 along a y-axis. The second arm 116 may include an opening or ledge (hereinafter “opening”) 115 embedded within a second arm 116, at or near a top surface of the second arm 116. Embedded within the first hinge 114 may be a gear 140 such as a ratchet gear, a lever 142, a cam 143, a first catch or pawl (hereinafter “first pawl”) 144, a first spring 145 associated with the first pawl 144, a second catch or pawl (hereinafter “second pawl”) 146, and a second spring 147 associated with the second pawl 146.

The ledge 115 may be slid, translated, or moved along the z-axis. The ledge 115 may contact the gear 140, thus spinning the gear in a counterclockwise direction. The ledge 115 may click or stop at discrete points due to the first pawl 144 that engages or interfaces with the teeth of the gear 140 at discrete intervals defined by width of the teeth of the gear 140. As the gear 140 rotates, the first pawl 144 permits slippage of the gear 140, and terminates the slippage at discrete points. The first pawl 144 rests onto each step of the gear 140 on a rim of the gear 140, thereby constraining further movement of the gear 140, and of the ledge 115, at discrete points. Therefore, a position of the device 100, in particular a torsion angle of the device 100 to implement asymmetric compression, may be temporarily locked and/or maintained, instead of the ledge 115 being continuously sliding. The second pawl 146 may engage the teeth of the gear 140 and prevent the gear 140 from turning in a reverse, or clockwise, direction. Meanwhile, a switch within the cam 143 may change an orientation of the first pawl 144 and the second pawl 146. The switch may be used to reverse the orientation or direction of the gear 140 and therefore move the ledge along the negative z-axis instead. Meanwhile, the fourth arm 126 may be translated with respect to the third hinge 124 in a same or similar manner as that described with respect to the second arm 116.

FIG. 1G illustrates a mechanism via which the first arm 112 may rotate about a y-axis with respect to the main body 110. For example, a portion of the first arm 112 that extends over, or overlaps, the main body 110, may include openings or holes 152, 153, 154, and 155 on a front surface and corresponding openings or holes 162, 163, 164, and 165 on a back surface. The main body 110 may include any number of holes. In a straight, unrotated position, a fastener may pass through the hole 152, then a hole on the main body 110, and the hole 162. In a rotated position, a fastener may pass through the hole 152, then a hole on the main body 110, and the hole 163. Thus, the first arm 112 may be in a rotated orientation with respect to the main body 110.

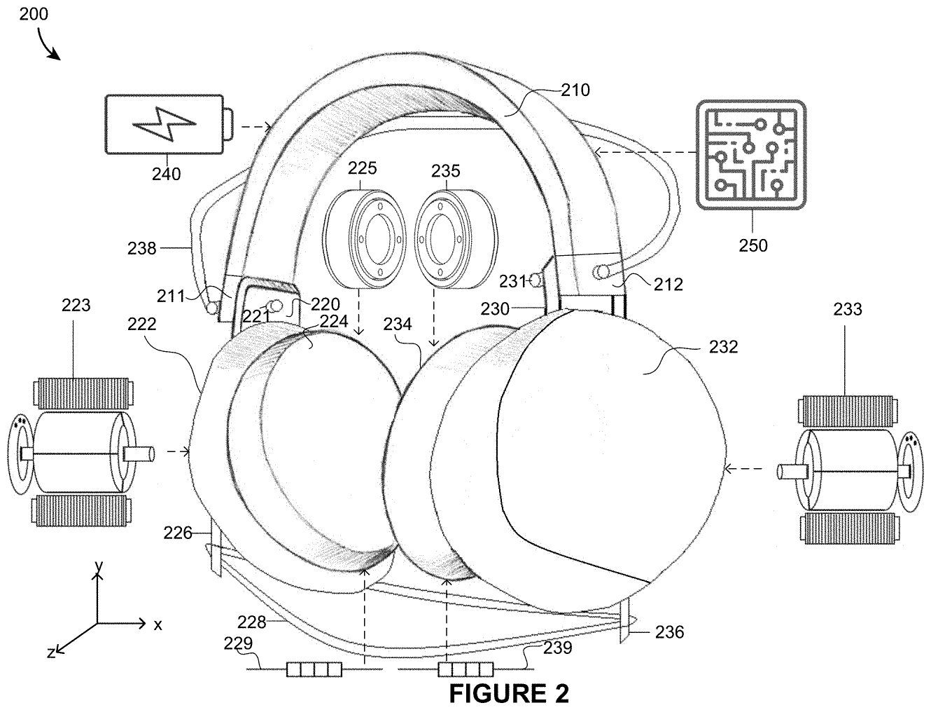

FIG. 2 illustrates an exemplary device 200 that relieves pain and/or implements a therapeutic treatment, in accordance with an embodiment. In some embodiments, the device 200 may be implemented as a clamp or a brace. In FIG. 2, the device 200 includes a band or main body (hereinafter “main body”) 210, a first hinge or first arm (hereinafter “first arm”) 220 connected to the main body 210 via a first fastener 221, a first terminal portion, a second hinge or second arm (hereinafter “second arm”) 230 connected to the main body 210 via a second fastener 231, and a second terminal portion. In some embodiments, as illustrated in FIG. 2, the first terminal portion includes plates or pads (hereinafter “plate(s)”) 222 and/or 224 (hereinafter “222, 224” when referred to collectively) and the second terminal portion includes plates 232 and/or 234 (hereinafter “232, 234” when referred to collectively). The plates 222, 224 may directly contact each other, and/or may be integrated. Similarly, the plates 232, 234 may directly contact each other and/or be integrated. Although the plates 222, 224 and 232, 234 are shown as circular or elliptical shaped, the plates 222, 224 and 232, 234 may also be elliptical or rectangular (e.g., square) shaped. In other embodiments, instead of the plates 222, 224, the first and second terminal portions may include cups such as suction cups, or chambers, compartments or containers, and/or adapters that are configured to interface or attach to the cups, chambers, compartments, or containers.

The main body 210 may be made from any of injection molded plastics or metals such as cast alloys and sheet metals. The main body 210 may be flexible to accommodate different orientations or positions of the terminal portions. For example, the plates 222, 224 and 232, 234 may be rotated about any of the x, y, and/or z-axes relative to each other as a result of the rotation of the main body 210. The plates 222, 224 and 232, 234 may be rotated to a twist angle or a torsion angle of between 20 and 45 degrees relative to each other. Applying therapeutic treatment at a twist or torsion angle may facilitate or cause the release of endorphins at an accelerated rate compared to an application of therapeutic treatment with no twist or torsion. The main body 210 may be arcuate, U-shaped, or flat-sectioned.

Further attached to the main body 210 may be a battery 240. The battery 240 may be a lithium ion battery and/or have a maximum capacity of between 4.2 Volts and 12 Volts. In some embodiments, the battery 240 may be between 2000 mAh (milliamp hours) and 20000 mAh.

The first arm 220 may be slid into a groove of the plate 222 so that the plate 222 may be allowed to slide, relative to the y-axis, up and down the first arm 220. In some embodiments, the plate 222 may be allowed to slide, with respect to the first arm 220, along a x-axis and/or a z-axis direction. When the plate 222 is at a desired location, the plate 222 may be locked into place relative to the first arm 220. In some embodiments, the first arm 220 may be part of a pivot assembly, so that the plates 222, 224 can pivot or slide relative to the y-axis, or rotate about the x-axis, relative to the main body 210.

A heat source 229 may be embedded in, attached to, or associated with the plate 224. The heat source 229 may be adjustable to provide variable amounts of heat to a body surface. In other embodiments, the heat source 229 may be embedded within the plate 222. Optionally embedded within the plate 222, or otherwise attached to or associated with the plate 222, may be one or more motors 223 that, when actuated, can provide a rotary and/or linear actuation to massage, vibration, and/or compressive force, onto the plate 224 and/or the plate 222. The one or more motors 223 may be brushless DC motors and may be rotary or linear motors to actuate rotational or linear displacement onto the plate 224 and/or the plate 222 that is applied to a body surface. The one or more motors 223 may have noise reduction features that reduce noise to below 60 dB. In order to provide ventilation to the one or more motors 223, the plate 224 may include pores or grating. Also embedded in, attached to, or associated with the plate 224 may be a force transducer 225, such as a strain gauge, that detects an amount of force onto the plate 224 that contacts a body surface. Thus, the force transducer 225 may detect a non-zero force, or a force that is above a threshold, to determine when the body surface has contacted the plate 224. If the force is zero, then no body surface has contacted the plate 224. Optionally, also embedded within any of the plates 222, 224, and/or 232, 234 may be a static magnet to apply magnetic therapy.

In some embodiments, the device 200 may be manually operated. In other embodiments, some functions of the device 200 may be electronically controlled. Optionally, the device 200 may include a processor 250 programmed to acquire sensor data of the body surfaces and perform some functions of implementing a therapeutic treatment at the plates 224 and 234, as will be explained further. Although the processor 250 is illustrated as a single component, the processor 250 may refer collectively to all entities that may perform processing functions to operate the device 200. A reading of the force from the force transducer 225 may be transmitted to the processor 250, which may commence a therapeutic treatment upon detecting a non-zero force, or a force that is above a threshold. Thus, once the force transducer 225 determines that the body surface has contacted the plate 224, the processor 250 may switch on at least a portion of the one or more motors 223.

Similar to the first arm 220, the second arm 230 may be slid into a groove of the plate 232 so that the plate 232 may be allowed to slide, relative to the y-axis, up and down the second arm 230. In some embodiments, the plate 232 may be allowed to slide, with respect to the second arm 230, along a x-axis and/or a z-axis direction. When the plate 232 is at a desired location, the plate may be locked into place relative to the second arm 230. In some embodiments, the second arm 230 may be part of a pivot assembly, together with a member 2212, so that the plates 232 and 234 can pivot, or rotate about the x-axis, relative to the main body 210. Embedded within the plate 232 may be one or more motors 233 that, when actuated, can provide a rotary and/or linear actuation to massage, vibration, and/or compressive force, onto the plate 234. The one or more motors 233 may be brushless DC motors and may be rotary or linear motors to actuate rotational or linear displacement onto the plate 234 that is applied to a body surface. The one or more motors 233 may have noise reduction features that reduce noise to below 60 dB. In order to provide ventilation to the one or more motors 233, the plate 234 may include pores or grating. The plate 234 may further have an embedded heat source 239 having adjustable settings to provide variable amounts of heat to a body surface. In other embodiments, the heat source 239 may be embedded within the plate 232. Also embedded in the plate 234 may be a force transducer 235, such as a strain gauge, that determines an amount of force applied onto the plate 234 by a body surface. Thus, the force transducer 235 may detect a non-zero force, or a force that is above a threshold, to determine when the body surface has contacted the plate 234. A reading from the force transducer 235 may be transmitted to the processor 250, which may commence a therapeutic treatment upon detecting a non-zero force, or a force that is above a threshold. Thus, once the force transducer 235 determines that the body surface has contacted the plate 234, the processor 250 may switch on at least a portion of the one or more motors 233.

In some embodiments, a hardness of the plate 234 may differ from that of the plate 224 so that the plates 224 and 234 may provide different therapeutic treatments. For example, the plate 234 may be harder and/or coarser compared to the plate 224. In some examples, the plate 234 may be made of silicone, while the plate 224 may be made of a foam such as urethane foam. In some embodiments, both the plates 224 and 234 have a slight concavity to fit body surfaces. In some embodiments, the plates 222 and 232 may be harder compared to the plates 224 and 234. The plates 222 and 232 may be made from any suitable materials, for example, nylon or glass fiber.

To further secure or clamp the device 200 to a body surface, a cord or band 228 may encircle a different body surface. The cord or band 228 may be tensioned and/or elastic, and may be wrapped around one or both of members 226 and 236, which extend from the plates 222 and 232, respectively. Optionally, a strap 238 may wrap around an other body surface. For example, if at least one of the plates 224 and 234 is positioned to contact a back of a person or other organism, the strap 238 may be wrapped around a front portion of the person or other organism. The strap 238 may extend from near one terminal portion of the device 200 to an other terminal portion of the device 200, and may lock, click, or otherwise be secured into place at one of the terminal portions of the device 200. In some examples, principles illustrated with respect to FIGS. 1A-1G and FIG. 2 may be combined in a single device or embodiment.

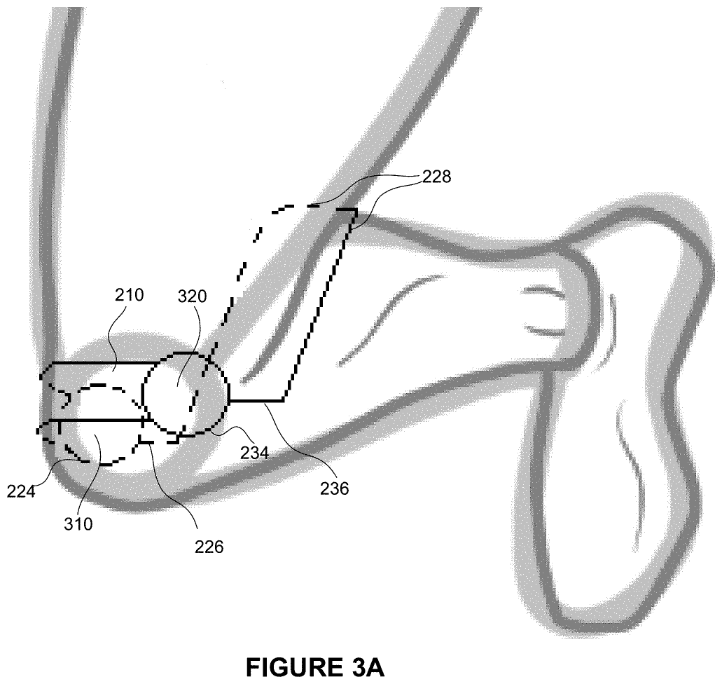

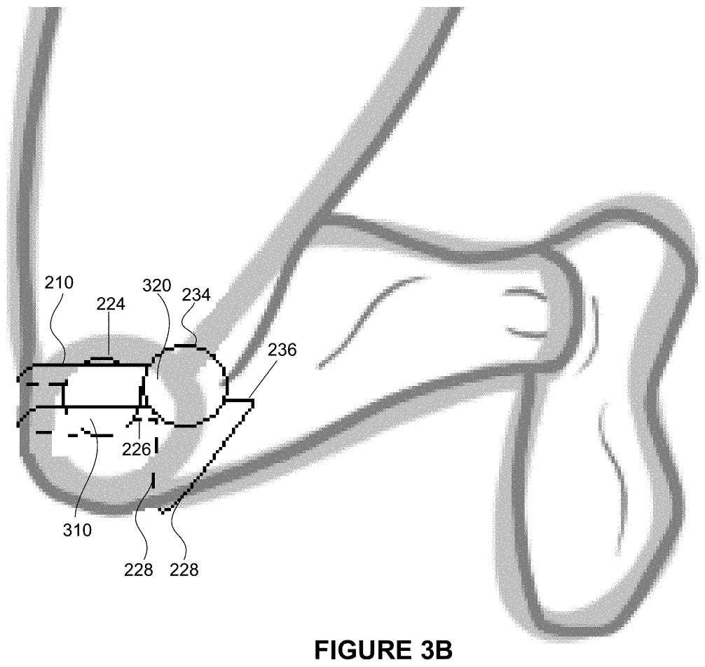

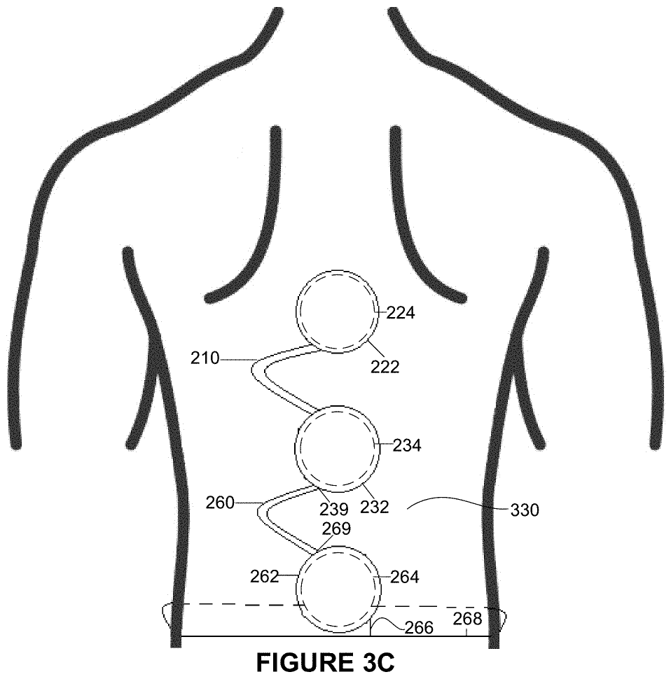

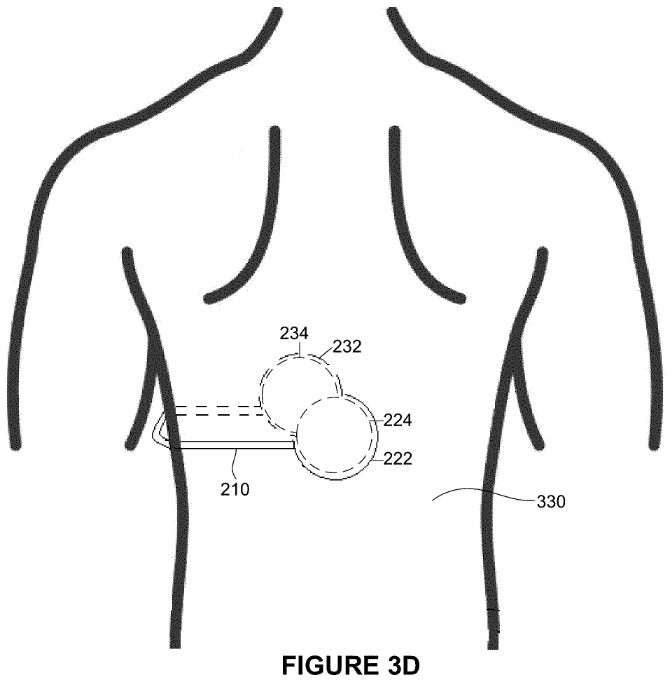

FIGS. 3A-3C illustrate exemplary implementations of the device 200 or of the device 100, in accordance with an embodiment. In some embodiments, the device 200 or the device 100 may fit over an existing compressive brace, such as a knee, shoulder, or lower back brace, that has openings to fit haptic and/or heat modules. Although FIGS. 3A-3C refer to components of FIG. 2, FIGS. 3A-3C may also be applicable to FIGS. 1A-1E. In FIG. 3A, the plates 224 and 234 may contact two opposing body surfaces 310 and 320, respectively, around a knee of a person, while the main body 210 extends over the knee. The band 228 may extend between the members 226 and 236 while snugly encircling a back portion of a lower leg, in other words, a region behind the device 200, to affix or clamp the device around the knee. In FIG. 3B, the band 228 may instead snugly encircle a front portion of the lower leg, in other words, a region in front of the device 200. Either one or both of the band 228 and the strap 238 may be utilized. In some embodiments, the band 228 may wrap around the back portion of the lower leg in addition to the strap 238 encircling the front portion of the lower leg. FIG. 3C illustrates the device 200 being positioned to contact a back 330 of a human. In particular, the plates 224 and 234 may contact different areas of the back 330. The plates 222 and 232 may be directly over the plates 224 and 234. The device 200 may further include additional portions, or modules, such as, an additional plates 262 and 264 which are connected to the plate 232 by an additional hinge 260. Any number of hinges and plates may be included in the device 200. The additional hinge 260 may be connected to the plate 232 at an attachment 239 via mechanical fitting and/or magnetic fitting. The attachment 239 may be an opening or a groove. Similarly, the additional hinge 260 may be connected to the plate 262 at an attachment 269 via mechanical fitting and/or magnetic fitting. A cord 268 connected to a member 266 extending from the plate 262, similar to the cord 228, may wrap snugly around a front surface opposite the back 330 so that the additional plate 264 is further secured onto the back 330. The cord 268 may lock, click, or otherwise be secured into place at the member 266. Additional cords may wrap around a front surface to further secure the plates 234 and 224 to the back 330. FIG. 3D illustrates the device 200 being secured to the back 330 using an alternative configuration from FIG. 3C. In particular, in FIG. 3D, In particular, the plates 222, 224 may contact the back 330 while the plates 232, 234 may contact a front of a human body around a stomach area. The main body 210 may be wrapped from the back 330 to the front of the human body.

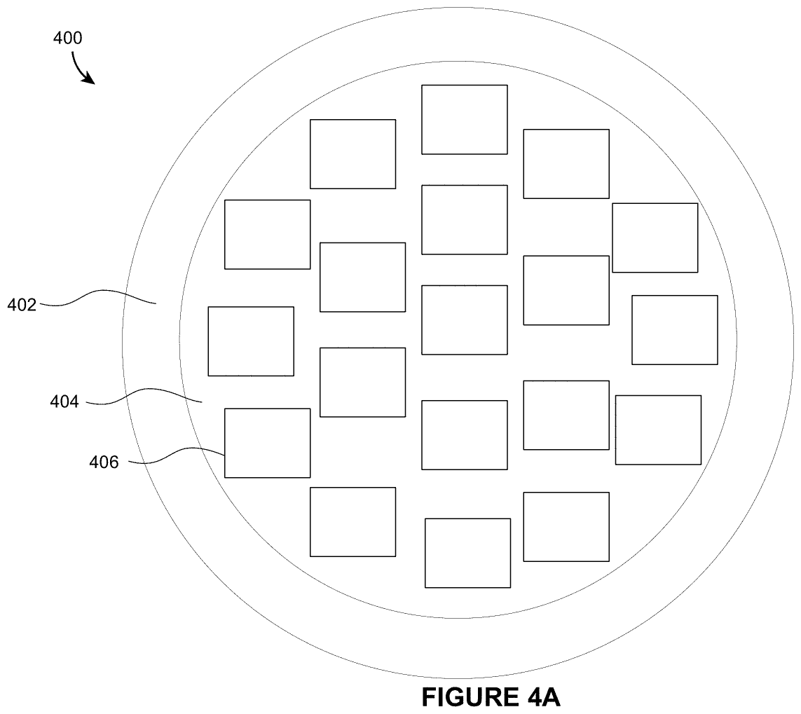

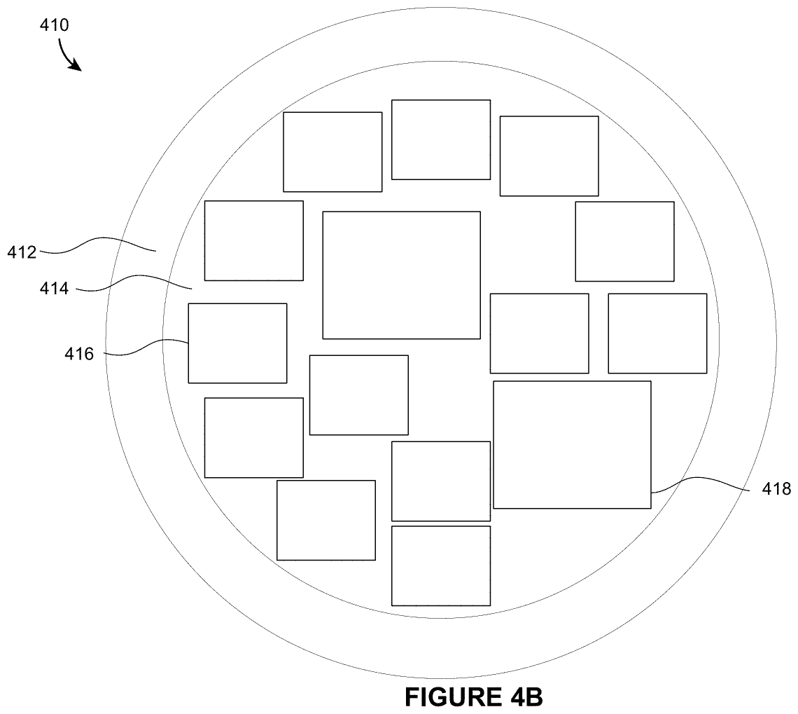

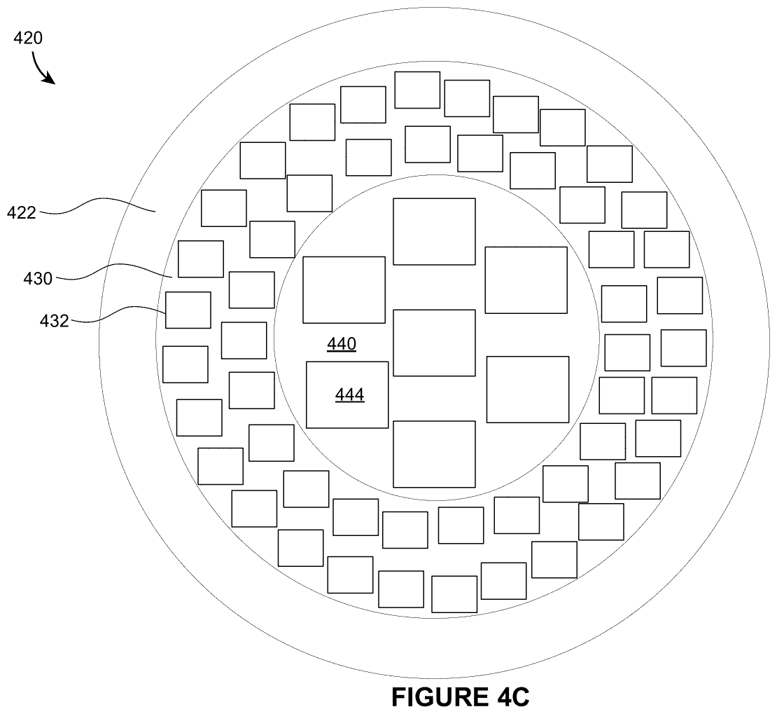

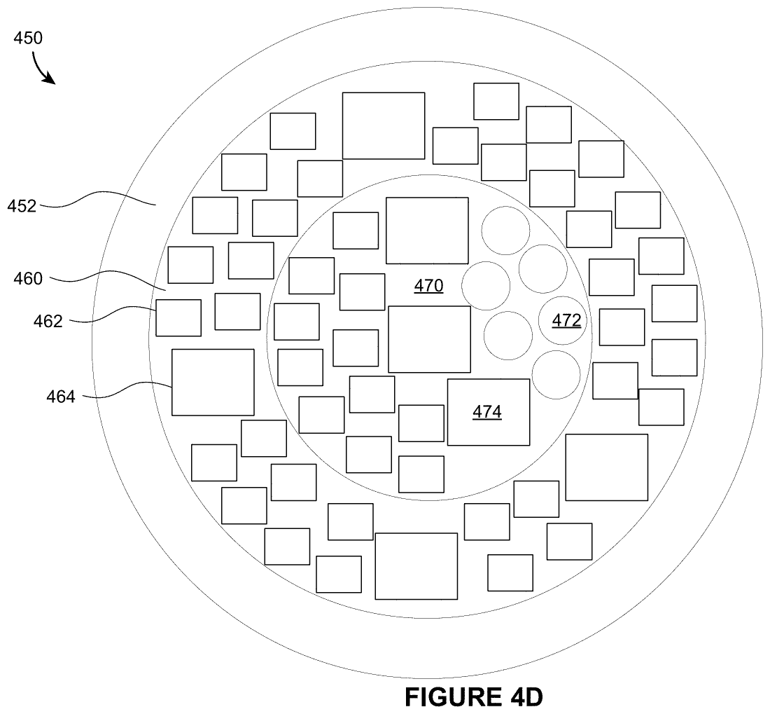

FIG. 4A illustrates an exemplary implementation of a plate 400, which may be implemented as the plate 224 or 234, or the plate 120 or 130, in accordance with an embodiment. The plate 400 may include a periphery 402 to increase adhesion to a body surface. The periphery 402 may have a higher friction compared to an interior 404. The interior 404 may include beads 406. Although the beads 406 are illustrated as rectangular or square shaped, the beads 406 may have other shapes, such as ellipsoids, spheres, circles or ellipses. In some examples, the beads 406 may be wooden. Optionally, the beads 406 may be rotated and/or linearly actuated by the one or more motors 223 or 233, as controlled by the processor 250, to provide a massage effect. A direction of linear actuation may be horizontal and/or vertical. A direction of rotation and/or linear actuation may be alternated or changed, for example, at periodic intervals. A linear velocity, linear acceleration, angular velocity, and/or angular acceleration may also be changed, for example, at periodic intervals.

FIG. 4B illustrates an exemplary implementation of a plate 410, which may be implemented as the plate 224 or 234, or as the plate 120 or 130, in accordance with an embodiment. The plate 410 may include a periphery 412 to increase adhesion to a body surface. The periphery 412 may have a higher friction compared to an interior 414. The interior 414 may include small beads 416 and large beads 418. Although the small beads 416 and large beads 418 are illustrated as rectangular or square shaped, the small beads 416 and large beads 418 may have other shapes, such as ellipsoids, spheres, circles or ellipses. Thus, unlike the plate 400 of FIG. 4A, the plate 410 may have variable sized beads. In some examples, the small beads 416 and the large beads 418 may be wooden. The small beads 416 and the large beads 418 may be rotated and/or linearly actuated by the one or more motors 223 or 233, as controlled by the processor 250, to provide a massage effect. A direction of linear actuation may be horizontal and/or vertical. A direction of rotation and/or linear actuation may be alternated or changed, for example, at periodic intervals. A linear velocity, linear acceleration, angular velocity, and/or angular acceleration may also be changed, for example, at periodic intervals. In some examples, the plate 410 may have beads of three or more different sizes.

FIG. 4C illustrates an exemplary implementation of a plate 420, which may be implemented as the plate 224 or 234, or as the plate 120 or 130, in accordance with an embodiment. The plate 420 may include a periphery 422 to increase adhesion to a body surface. The periphery 422 may have a higher friction compared to an interior which may include regions 430 and 440. The region 430 may include small beads 432 and the region 440 may include large beads 444. Thus, unlike the plate 400 of FIG. 4A, the plate 420 may have variable sized beads separated by regions. Although the small beads 432 and large beads 444 are illustrated as rectangular or square shaped, the small beads 432 and large beads 444 may have other shapes, such as ellipsoids, spheres, circles or ellipses. In some examples, the small beads 432 and the large beads 444 may be wooden. The small beads 432 and the large beads 444 may be rotated and/or linearly actuated by the one or more motors 223 or 233, as controlled by the processor 250, to provide a massage effect. A direction of linear actuation may be horizontal and/or vertical. A direction of rotation and/or linear actuation may be alternated or changed, for example, at periodic intervals. A linear velocity, linear acceleration, angular velocity, and/or angular acceleration may also be changed, for example, at periodic intervals. A direction of rotation and/or linear actuation may be controlled to be opposite or different between the regions 430 and 440. Additionally, a linear velocity, linear acceleration, angular velocity, and/or angular acceleration may also be controlled to be different between the regions 430 and 440. In some examples, the small beads 432 in the region 430 may be initially rotated in a counterclockwise direction while the large beads 444 in the region 440 may be initially rotated in a clockwise direction. The directions of rotation of the small beads 432 in the region 430 and/or the large beads 444 in the region 440 may be alternated, for example, periodically. Thus, after a periodic interval, the small beads 432 in the region 430 may be rotated in a clockwise direction while the large beads 444 in the region 440 may be rotated in a counterclockwise direction. Although not shown, in other examples, the region 430 may include large beads while the region 440 may include small beads. In some examples, the plate 420 may have beads of three or more different sizes and/or three or more different regions. Nearest adjacent regions may have opposite directions of rotation or directions of actuation.

FIG. 4D illustrates an exemplary implementation of a plate 450, which may be implemented as the plate 224 or 234, or as the plate 120 or 130, in accordance with an embodiment. The plate 450 may include a periphery 452 to increase adhesion to a body surface. The periphery 452 may have a higher friction compared to an interior which may include regions 460 and 470. The region 460 may include small beads 462 and large beads 464, while the region 470 may include small beads 472 and large beads 474. Although the small beads 462 and large beads 464 are illustrated as rectangular or square shaped, the small beads 462 and large beads 464 may have other shapes, such as ellipsoids, spheres, circles or ellipses. Although the small beads 472 and large beads 474 are illustrated as rectangular or square shaped, the small beads 472 and large beads 474 may have other shapes, such as ellipsoids, spheres, circles or ellipses. Thus, unlike the plate 420 of FIG. 4C, the plate 450 may have different sized beads in each region. In some examples, the small beads 462 and 472, and the large beads 464 and 474 may be wooden. The small beads 462 and 472 and the large beads 464 and 474 may be rotated and/or linearly actuated by the one or more motors 223 or 233, as controlled by the processor 250, to provide a massage effect. A direction of linear actuation may be horizontal and/or vertical. A direction of rotation and/or linear actuation may be alternated or changed, for example, at periodic intervals. A linear velocity, linear acceleration, angular velocity, and/or angular acceleration may also be changed, for example, at periodic intervals. A direction of rotation and/or linear actuation may be controlled to be opposite or different between the regions 460 and 470. Additionally, a linear velocity, linear acceleration, angular velocity, and/or angular acceleration may also be controlled to be different between the regions 460 and 470. In some examples, the small beads 462 and the large beads 464 in the region 460 may be initially rotated in a counterclockwise direction while the small beads 472 and the large beads 474 in the region 470 may be initially rotated in a clockwise direction. The directions of rotation of the small beads 462 and the large beads 464 in the region 460 and/or the small beads 472 and the large beads 474 in the region 470 may be alternated, for example, periodically. Thus, after a periodic interval, the small beads 462 and the large beads 464 in the region 460 may be rotated in a clockwise direction while the small beads 472 and the large beads 474 in the region 470 may be rotated in a counterclockwise direction. In some examples, the plate 450 may have beads of three or more different sizes and/or three or more different regions. Nearest adjacent regions may have opposite directions of rotation or directions of actuation.

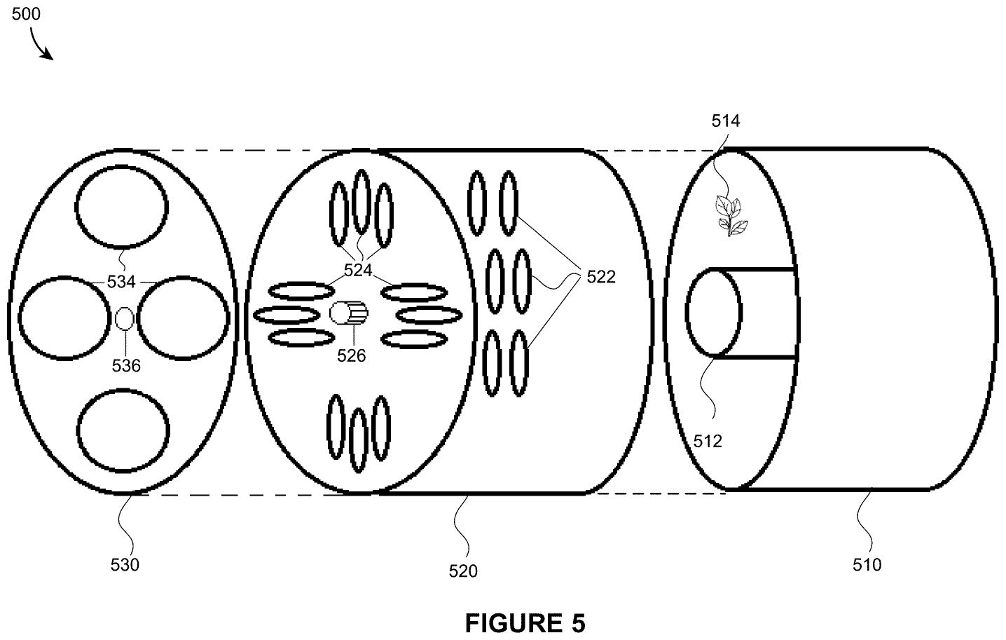

FIG. 5 illustrates an implementation used in conjunction with the device 200, or the device 100, in accordance with an embodiment. In FIG. 5, a chamber, compartment, or container 500 may be implemented in place of the plate 224 and/or the plate 234, or in place of the plate 120 or 130. The chamber 500 may include a first portion 510 which may include a heat source 512 and a plant extract 514. In some embodiments, plant extract 514 may be integrated with the heat source 512. The chamber 500 may include a second portion 520 that may slide over and/or attach to the first portion 510. The second portion may include pores or openings 522 on sides of the second portion 520 to allow heat to be transferred out of the chamber 500. The pores 522 may be fixed in size and perpetually open. Meanwhile, pores or openings 524 on a top surface of the second portion 520 may be adjustable in size via rotation of a grating 530 to be attached over the second portion 520. The grating 530 may include pores or openings 534 and a hole 536. A fastener 526 of the second portion 520 may be attached through the hole 536. The grating 530 may be positioned over and rotated with respect to the second portion 520 to regulate sizes of the openings 524, and in turn, an amount of heat transmitted through the chamber 500. For example, if positions of the pores or openings 534 coincide with positions of the pores or openings 524, then a total surface area of the openings 524 is at a maximum. To completely close or minimize a size of the pores or openings 524, the pores or openings 534 may be positioned 45 degrees with respect to the pores or openings 524. As the chamber 500 may be in contact with the plate 222 and/or 232, the one or more motors 223 and/or 233 may be implemented to rotate the grating 530, as controlled by the processor 250, in order to adjust an amount of heat transmitted from the chamber 500. In some embodiments, the chamber 500 may be enclosed within a velvet pouch to prevent surfaces of the chamber from directly contacting and burning skin. In some embodiments, the chamber 500 may be a moxibustion chamber.

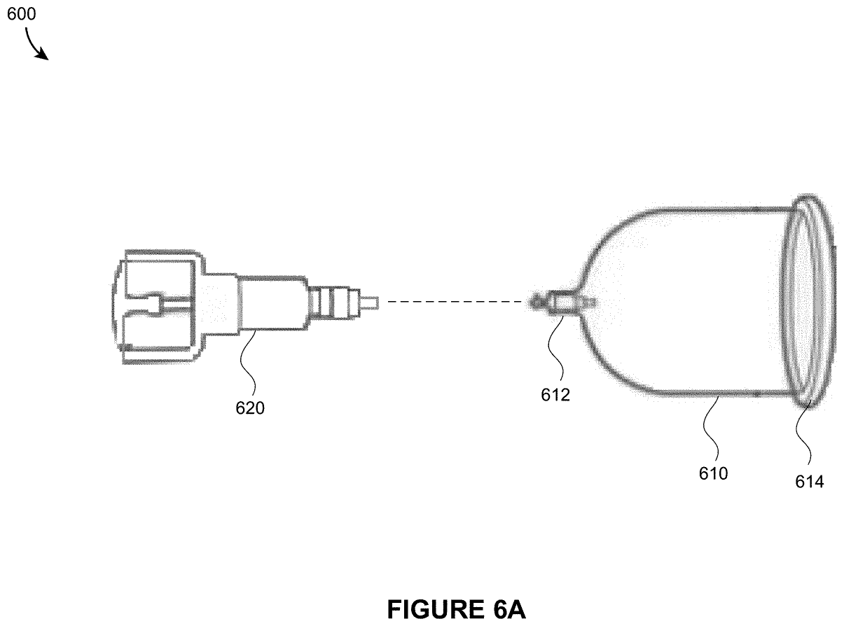

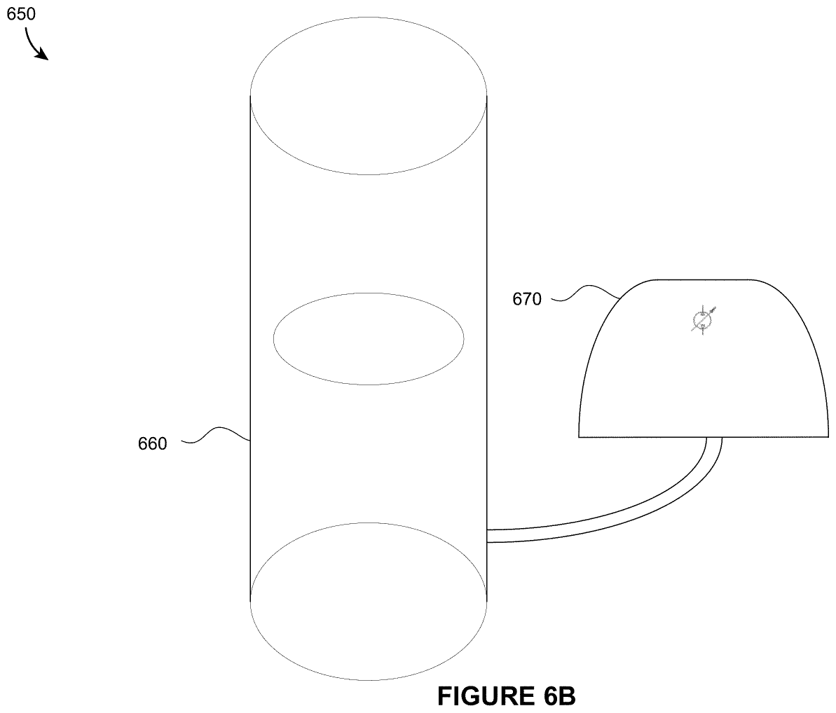

FIG. 6A illustrates an implementation 600 used in conjunction with the device 200, or the device 100, in accordance with an embodiment. In FIG. 6A, a cup 610 such as a suction cup may be implemented in place of the plate 224 and/or the plate 234, or in place of the plate 120 and/or 130. The cup 610 may include a port 512 to which a pump 620 may be attached to pressurize a space inside the cup 610 when a surface 514 is pressed or sealed onto a body surface. The pump 620 may be actuated by the one or more motors 223 and/or 233, as controlled by the processor 250, to adjust an amount of suction pressure inside the cup 610. The pump 620 may be embedded within the plates 224 and/or 234, or otherwise coupled, directly or indirectly, to the plates 224 and/or 234. In some examples, the one or more motors 223 and/or 233 may apply a linear actuation to push a plunger or piston of the pump 620. When linear actuation is applied to the pump 620, pressure is added inside the cup 610.

FIG. 6B illustrates an implementation 650 used in conjunction with the device 200, in accordance with an embodiment. In FIG. 6B, a pneumatic compressor 660 may be connected to the plate 224 and/or the plate 234, or, or the plate 120 or 130, to wrap securely around a body part, such as a leg, knee, or other limb. In some examples, the plate 224 and/or the plate 234 may slide or move along, or be positioned somewhere on a surface of the pneumatic compressor 660 at a particular location at which therapeutic treatment is applied. The pneumatic compressor may be connected to a pneumatic pump 670 which may be a variable displacement pump. The pneumatic pump 670 may apply variable amounts of pressure.

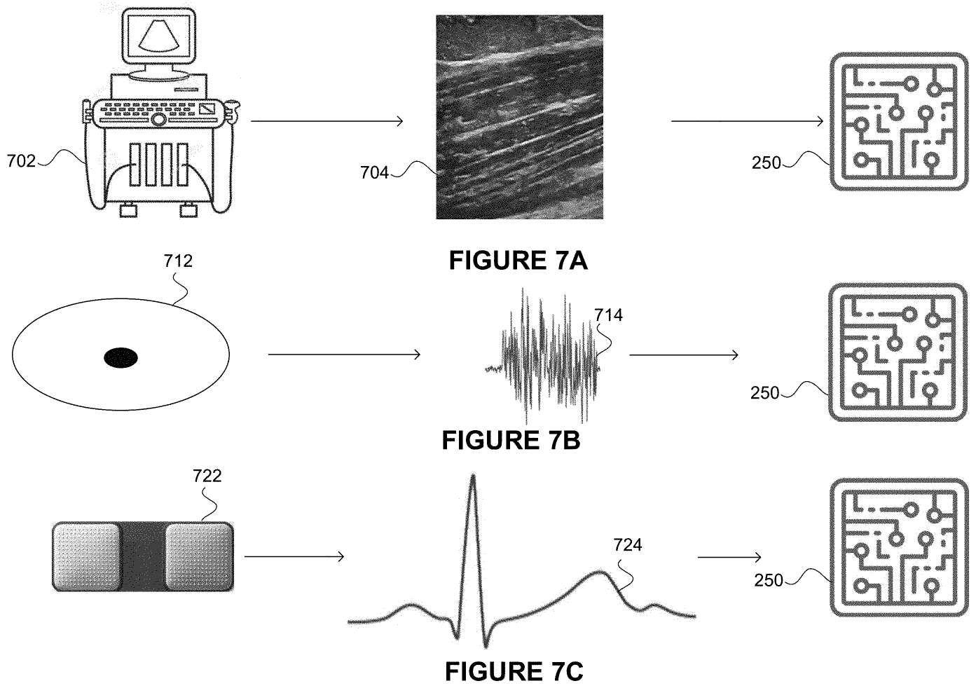

FIGS. 7A-7C illustrate implementations of a processor, such as the processor 250, in accordance with an embodiment. Generally, the processor 250 may receive input regarding parameters of the application of therapeutic treatment, including, but not limited to, vibration frequencies, vibration styles (for example, oscillation vibration, spiral vibration, or triplanar vibration simultaneously or sequentially), rotation directions, rotation speeds, linear actuation directions, linear actuation speeds, magnitude and duration of compression and/or heating, duration and at different tissues such as joints. The processor 250 may be trained, for example, using machine learning, to implement particular parameters of therapeutic treatment, or a range of parameters, at particular tissues such as joints. In some examples, the processor 250 may be trained over time using input of particular parameters of actual therapeutic treatment from different users under different conditions. The different conditions may indicate an existence and degree of tissue injury and/or fatigue, mass and/or volume of particular tissues, and other tissue parameters, and how parameters of actual therapeutic treatment may vary under these different conditions. Upon receiving input, determining, or estimating the different conditions, the processor 250 may implement or suggest a particular therapeutic treatment to be implemented. The processor 250 may further learn over time based on user input and/or an efficacy of the particular therapeutic treatment. For example, if, following the suggestion by the processor 250, a user selects a different therapeutic treatment, the processor 250 may adapt so that during a subsequent cycle under similar or same conditions, the processor 250 may implement or suggest a therapeutic treatment closer to or same as the user-selected therapeutic treatment. In some examples, if the efficacy of the particular therapeutic treatment selected and actually implemented by the processor 250 fails to satisfy a threshold efficacy or condition, the processor 250 may adjust one or more parameters during a subsequent cycle under similar or same conditions. The processor 250 may determine or estimate the efficacy based on physiological signals such as EMG (electromyography) or ECG (electrocardiogram) signals.

In FIG. 7A, the processor 250 may acquire image data 704, for example, from an imaging machine 702 such as an ultrasound, MRI (magnetic resonance imaging), or CT (computerized tomography) machine. The processor 250 may, from the image data, identify types of tissues. The processor 250 may be trained to distinguish different tissues and boundaries of tissues using semantic segmentation and/or instance segmentation. In some examples the processor 250 may be trained to analyze and/or infer parameters or properties of tissue, such as, for example, an existence and degree of tissue injury and/or fatigue, mass and/or volume of particular tissues. The processor 250 may, based on the identified types or tissues and/or determined tissue properties, determine a particular protocol of therapeutic treatment. For example, if the processor 250 determines that a particular muscle is injured or strained, the processor 250 may control an amount of heat to be reduced or eliminated, and/or apply a steady, low amount of compression.

In FIG. 7B, the processor 250 may acquire EMG data 714 from an EMG sensor 712 and adjust one or more parameters of the therapeutic treatment based on the EMG data 714, either during a current cycle or in a subsequent cycle. The processor 250 may be trained to interpret the EMG data 714, from EMG training data that includes particular scenarios such as muscle injury, damage, and/or abnormal activity so that the processor 250 can recognize or infer different muscle conditions from the EMG data 714. For example, if the processor 250 infers, from the EMG data 714, abnormal electrical activity and/or muscle damage, the processor 250 may accordingly adjust a protocol of the therapeutic treatment. For example, the processor may control a force applied by a massage treatment and/or a compression to be reduced, and/or a duration of the therapeutic treatment to be increased.

In FIG. 7C, the processor 250 may acquire ECG data 724 from an ECG sensor 722 and adjust one or more parameters of the therapeutic treatment based on the ECG data 722, either during a current cycle or in a subsequent cycle. The processor 250 may be trained to interpret the ECG data 724, from ECG training data that includes particular scenarios such as elevated heart rate or abnormal heart rhythm so that the processor 250 can recognize or infer different heart conditions from the EMG data 714. For example, if the processor 250 infers, from the ECG data 724, an elevated heart rate or abnormal heart rhythm, the processor 250 may accordingly adjust a protocol of the therapeutic treatment. For example, the processor may control a force applied by a massage treatment and/or a compression to be reduced, and/or a magnitude of heat to be decreased.

The techniques described herein, for example of the processor 250, are implemented by one or more special-purpose computing devices. The special-purpose computing devices may be hard-wired to perform the techniques, or may include circuitry or digital electronic devices such as one or more application-specific integrated circuits (ASICs) or field programmable gate arrays (FPGAs) that are persistently programmed to perform the techniques, or may include one or more hardware processors programmed to perform the techniques pursuant to program instructions in firmware, memory, other storage, or a combination.

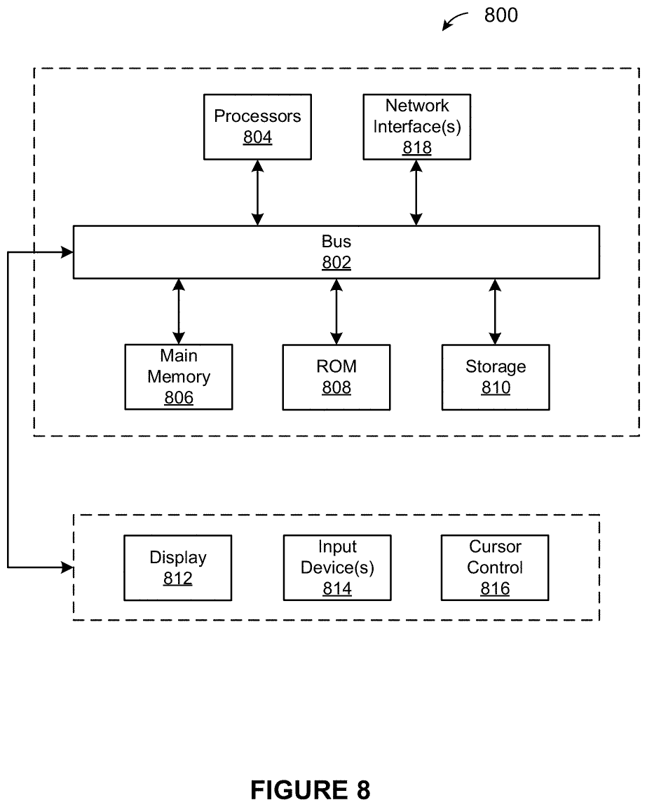

FIG. 8 illustrates a block diagram of a computer system 800 upon which any of the embodiments described herein may be implemented. The computer system 800 includes a bus 802 or other communication mechanism for communicating information, one or more hardware processors 804, which may be implemented as the processor 250, coupled with bus 802 for processing information. A description that a device performs a task is intended to mean that one or more of the hardware processor(s) 804 performs.

The computer system 800 also includes a main memory 806, such as a random access memory (RAM), cache and/or other dynamic storage devices, coupled to bus 802 for storing information and instructions to be executed by processor 804. Main memory 806 also may be used for storing temporary variables or other intermediate information during execution of instructions to be executed by processor 804. Such instructions, when stored in storage media accessible to processor 804, render computer system 800 into a special-purpose machine that is customized to perform the operations specified in the instructions.

The computer system 800 further includes a read only memory (ROM) 808 or other static storage device coupled to bus 802 for storing static information and instructions for processor 804. A storage device 810, such as a magnetic disk, optical disk, or USB thumb drive (Flash drive), etc., is provided and coupled to bus 802 for storing information and instructions.

The computer system 800 may be coupled via bus 802 to output device(s) 812, such as a cathode ray tube (CRT) or LCD display (or touch screen), for displaying information to a computer user. Input device(s) 814, including alphanumeric and other keys, are coupled to bus 802 for communicating information and command selections to processor 1804. Another type of user input device is cursor control 816. The computer system 800 also includes a communication interface 818 coupled to bus 802.

Unless the context requires otherwise, throughout the present specification and claims, the word “comprise” and variations thereof, such as, “comprises” and “comprising” are to be construed in an open, inclusive sense, that is as “including, but not limited to.” Recitation of numeric ranges of values throughout the specification is intended to serve as a shorthand notation of referring individually to each separate value falling within the range inclusive of the values defining the range, and each separate value is incorporated in the specification as it were individually recited herein. Additionally, the singular forms “a,” “an” and “the” include plural referents unless the context clearly dictates otherwise. The phrases “at least one of,” “at least one selected from the group of,” or “at least one selected from the group consisting of,” and the like are to be interpreted in the disjunctive (e.g., not to be interpreted as at least one of A and at least one of B).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiment.

A component being implemented as another component may be construed as the component being operated in a same or similar manner as the another component, and/or comprising same or similar features, characteristics, and parameters as the another component.

1. A device that applies therapeutic treatment to a subject, wherein the therapeutic treatment comprises any two modalities applied simultaneously, the modalities selected from compression, heat, vibration, and massage, the device comprising:
a main body;
hinges coupled to the main body on each side of the main body; and
respective plates connected to the hinges, wherein the respective plates are configured to contact opposing surfaces of a body.

2. The device of claim 1, wherein:
the respective plates comprise heat sources embedded within; and
the device applies heat and compression simultaneously.

3. The device of claim 2, wherein the heat sources provide heat at between 39 degrees and 42 degrees Celsius for periods of between five and ten minutes.

4. The device of claim 2, wherein the compression is applied at between 20 mm Hg and 120 mm Hg.

5. The device of claim 1, wherein the plates apply vibration simultaneously with heat, wherein the vibration is between 20 hz and 155 hz.

6. The device of claim 1, further comprising respective arms disposed between the hinges and the plates, wherein the arms are translatable with respect to the hinges in a direction perpendicular to an alignment of the device, so that when the arms are translated with respect to the hinges, the device applies asymmetric compression.

7. The device of claim 6, wherein:
the arms each comprise a ledge;
the hinges each comprise a gear embedded within;
each respective ledge contacts a gear and locks at discrete positions.

8. The device of claim 7, wherein the hinges each comprise a pawl embedded within, the pawl engaging teeth of the gear at discrete intervals.

9. The device of claim 1, wherein the device is torsioned at an angle of between 20 degrees and 45 degrees between the respective plates.

10. The device of claim 1, wherein the therapeutic treatment comprises compression, heat, vibration, and massage.

11. A method of applying a therapeutic treatment to a subject, wherein the therapeutic treatment comprises any two modalities applied simultaneously, the modalities selected from compression, heat, vibration, and massage, using a device that comprises a main body, hinges coupled to the main body on each side of the main body; and respective plates connected to the hinges, wherein the application of the therapeutic treatment comprises contacting the plates to opposing surfaces of a body.

12. The method of claim 11, further comprising:
generating heat from heat sources embedded within the device; and
the application of the therapeutic treatment comprises applying heat and compression simultaneously.

13. The method of claim 12, wherein the application of the therapeutic treatment comprises applying the heat at between 39 degrees and 42 degrees Celsius for period of between five and ten minutes.

14. The method of claim 12, wherein the application of the therapeutic treatment comprises applying the compression at between 20 mm Hg and 120 mm Hg.

15. The method of claim 11, wherein the application of the therapeutic treatment comprises applying heat simultaneously with vibration, wherein the vibration is between 20 hz and 155 hz.

16. The method of claim 11, wherein the therapeutic treatment comprises an asymmetric compression, and the application of the asymmetric compression comprises translating arms disposed between the hinges and the plates, the translation being in a direction perpendicular to an alignment of the device.

17. The method of claim 16, wherein the translation of the arms comprises contacting a gear with a ledge to lock the ledge at discrete positions, wherein the ledge is comprised within one of the arms and the gear is embedded within one of the ledges.

18. The method of claim 17, wherein the locking of the ledge comprises engaging, with a pawl, teeth of the gear at discrete intervals corresponding to the discrete positions, the pawl being embedded within the hinges.

19. The method of claim 11, further comprising torsioning the device at an angle of between 20 degrees and 45 degrees between the respective plates.

20. The method of claim 11, wherein the therapeutic treatment comprises compression, heat, vibration, and massage.
